(19) United States
(12) Patent Application Publication
(10) Pub. No.: US 2021/0015684 A1
(43) Pub. Date: Jan. 21, 2021

(54) Bamboo And Nonwoven Disposable Garments

(71) Applicant: Patricia Kayanga Nakabugo, Washington, DC (US)

(72) Inventor: Patricia Kayanga Nakabugo, Washington, DC (US)

(21) Appl. No.: 16/842,171

(22) Filed: Apr. 7, 2020

Related U.S. Application Data
(60) Provisional application No. 62/873,986, filed on Jul. 15, 2019.

(51) Int. Cl. A61F 13/496; A41B 17/00; A61F 13/472; A61F 13/49; A61F 13/53

(52) U.S. Cl. CPC A61F 13/496; A41B 17/00; A61F 13/472; A41B 9/001; A61F 13/53; A41B 2400/52; A61F 13/49017

(57) ABSTRACT

Undergarment products of the disclosure may comprise a bamboo fabric and a non-woven material. The undergarment may include bamboo fabric and a non-woven material in addition to other materials. In some cases, the garments may principally or primarily include bamboo fabric and a non-woven material with only the addition of further components and materials to manufacture the garment including stitching, elastic components, etc. In preferred embodiments, an undergarment of the disclosure may include a central section comprising bamboo fabric and side sections comprising a non-woven material. The bamboo used to make the underwear may be around 125 grams per square meter (GSM) and the non-woven material may be 100% cotton based.

### CROSS REFERENCE TO RELATED APPLICATIONS

This application claims priority under 35 USC § 119(e) to U.S. Provisional Application No. 62/873,986 filed Jul. 15, 2019, which is herein incorporated by reference in its entirety.

### BACKGROUND OF THE DISCLOSURE

### Field of the Invention

This disclosure generally relates to disposable undergarments including a traditional fabric such as bamboo, cotton, hemp, silk, wool, Tencel, lyocell/modal jute, ramie, and/or abaca and a nonwoven fabric such as cotton, rayon, bamboo, hemp, and silk.

### Description of the Related Art

Often when people hear of the disposable undergarments, they think of those directed to incontinence underwear, one example of which is Depends™. However, there are undergarments for individuals that use disposable underwear for non-incontinence purposes. Such garments are necessarily an adult diaper or leak resistant but can provide benefits. For example, a disposable underwear solution could be worn by a woman during her menstrual period. Ideally, such a product should include the use of sustainable materials. Traditional disposable undergarments are also not seamless (which is aesthetically attractive) which would allow a user to wear them with any piece of clothing and not have underwear lines.

Traditional disposable undergarments do not include bamboo or bamboo fabric and non-woven material. There are currently no available undergarments that include both bamboo fabric and a non-woven composition. Accordingly, there is a need for a disposable undergarment product, that is not necessarily an adult diaper product that is completely leak resistant, that can be made seamless and that can be comfortably worn in a variety of circumstances as dictated by the needs of the user.

### SUMMARY OF THE INVENTION

Undergarment products of the disclosure comprise a bamboo fabric and a non-woven material. The undergarment may include bamboo fabric and a non-woven material in addition to other materials. An undergarment of the disclosure may include a central section comprising bamboo fabric and side sections comprising a non-woven material. The bamboo used to make the underwear may be around 125 grams per square meter (GSM) and the non-woven material may be 100% cotton based.

The underwear can be made with or without a protective layer that may be used to protect against leakages. The protective layer can be in a form of a pad or leakage proof fabric in the mid-section between the pelvic/pubic area. Other features and aspects will be apparent from the following detailed description, the drawings, and the claims

### BRIEF DESCRIPTION OF THE DRAWINGS

FIG. 1A shows one embodiment of the front of an undergarment of the disclosure.

FIG. 1B shows one embodiment of the back of an undergarment of the disclosure.

FIG. 2A shows one embodiment of the front of an undergarment of the disclosure.

FIG. 2B shows one embodiment of the back of an undergarment of the disclosure.

FIG. 2C shows the back of an undergarment of the disclosure.

FIG. 3A shows the front of a boxer style undergarment of the disclosure.

FIG. 3B shows one embodiment of the back of a boxer style garment of the disclosure.

FIG. 4A shows one embodiment of the front of a high waist style undergarment of the disclosure.

FIG. 4B shows one embodiment of the back of a high waisted style undergarment of the disclosure.

FIG. 4C shows one embodiment of the front of a women's boy briefs style undergarment.

FIG. 4D shows one embodiment of the back of a women's boy briefs style undergarment.

FIG. 5A shows one embodiment of the front of a men's thong style undergarment.

FIG. 5B shows one embodiment of the back of a men's thong style undergarment.

FIG. 5C shows one embodiment of the front of a women's bikini style undergarment.

FIG. 5D shows one embodiment of the back of a women's bikini style undergarment.

FIG. 6A shows an embodiment of the front of a women's hipster style undergarment.

FIG. 6B shows an embodiment of the back of a women's hipster style undergarment.

FIG. 6C shows an embodiment of the front of a men's bikini style undergarment.

FIG. 6D shows an embodiment of the back of a men's bikini style undergarment.

FIG. 7 shows an embodiment of an undergarment with a protective layer.

Throughout the drawings and the detailed description, the same reference numerals refer to the same elements. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, products, and/or systems, described herein. However, various changes, modifications, and equivalents of the methods, products, and/or systems described herein will be apparent to an ordinary skilled artisan.

The presently claimed undergarments are an environmentally friendly hygienic option that differ from traditional disposable undergarments in that they can be made seamless in embodiments, they include bamboo fabric and a non-woven material and they feel as comfortable and breathable as any high end undergarment at an affordable cost.

The underwear combines two different types of fabric: traditional fabric and non-woven material. In this instance, traditional fabric refers to fabrics such as bamboo, cotton, hemp, silk, wool, Tencel, lyocell/modal jute, ramie, and/or abaca that may be eco-friendly. The traditional fabric (bamboo, cotton, hemp, silk, etc.) has elasticity and covers the front and back of the underwear and is connected by non-woven fabric on the sides. The fabric wicks away moisture but is not necessarily liquid absorbent. Accordingly, such fabric may or may not be used as a replacement for incontinency disposable underwear.

The nonwoven material may be virgin or blended to include materials such as: cotton, rayon, bamboo, hemp, and silk. The nonwoven fabric can be dry laid, air laid, spun bound or spun laced, and wet laid.

In embodiments, the non-woven fabric may be stitched on both sides to secure and connect the front end to the back end of the underwear so that it doesn't fall apart. The non-woven fabric can also be sewn in various other manners and styles as known in the art. Some of the underwear may be sewn to be seamless and other cuts may be made to have seams based on the style of underwear. Seamless underwear may be not stitched on the waistband and the outer base region on the sides that cover the rear and front region and legs. The stitching may be sewn in a matter that is not visible when worn. The underwear with stitching may also have a stylized designed stitch that incorporates, for example, a company brand as one example. Other like stitch patterns can be used as well. The style may be designed to fit different body sizes, shapes and types, and are sized to fit a broad range of users.

Bamboo fabric as used herein refers to a bamboo textile product. Bamboo textile can be cloth, yarn, or clothing made from bamboo fibers. Bamboo fiber may be used for a wide range of textile and fashion applications. Bamboo yarn may also be blended with other textile fibers such as hemp or spandex. Bamboo is renewable and can be replenished at a fast rate. Bamboo fibers are cellulose fibers extracted or fabricated from natural bamboo and vary widely.

Textiles made from bamboo may comprise a synthetic rayon made from cellulose extracted from bamboo. Bamboo is used whole and in strips; these strips may also be considered stiff fibers. Clothing comprising bamboo may also comprise a viscose rayon, a fiber made by dissolving cellulose in bamboo, and then extruding it to form fibers.

Bamboo is naturally resistant to pesticides which makes it an excellent product for this use. Additionally, the recovery time after harvest to plant new plants is much faster than, for example, cotton. Unlike cotton, bamboo stalks do not require much irrigation and can survive on natural rainfall. In fact, they can survive with natural rainfall. Preferably, the bamboo fiber can be made from 100% organic bamboo. Organic bamboo is highly absorbent, softer, and very breathable. Organic bamboo is odor resistant and wicks moisture. Organic bamboo easily accepts organic dyes. Organic bamboo plant grows 1 meter per day. Pesticides and fungicides are not needed for the its growth and the plants are seldom eaten by insects.

The bamboo fabric may range from about 100 grams per square meter (GSM) to about 500 GSM. The bamboo fabric may preferably be about 100 GSM to about 150 GSM; and even more preferably from about 115 GSM to about 135 GSM. The bamboo fabric is most preferably about 125 GSM. 125 GSM is lighter and not as thick as most standard underwear. Other materials that maintain the lightness and breathable feel of the fabric may also be used.

Non-woven fabric is a fabric-like material made from staple fiber (short) and long fiber (continuous long), bonded together by, for example, chemical, mechanical, heat or solvent treatment. The term can also be used in the textile manufacturing industry to denote fabrics which are neither woven nor knitted. Some non-woven materials lack sufficient strength unless densified or reinforced by a backing. In recent years, non-woven has also become an alternative to polyurethane foam.

Non-woven fabrics may be defined as sheet or web structures bonded together by entangling fiber or filaments (and by perforating films) mechanically, thermally or chemically. They may be flat or tufted porous sheets that are made directly from separate fibres, molten plastic or plastic film. They are not necessarily made by weaving or knitting and do not require converting the fibers to yarn. A certain percentage of recycled fabrics and oil-based materials may also be present in non-woven fabrics. The percentage of recycled fabrics vary based upon the strength of material needed for a specific use. In addition, some non-woven fabrics can be recycled after use, given the proper treatment and facilities. For this reason, some consider non-woven a more ecological fabric for certain applications, especially in fields and industries where disposable or single use products are important, such as hospitals, schools, nursing homes and luxury accommodations.

Nonwoven fabrics are generally engineered fabrics that may be single use, have a limited life, or be durable. Nonwoven fabrics may provide specific functions such as, but not limited to, absorbency, liquid repellence, resilience, stretch, softness, strength, flame retardancy, washability, cushioning, thermal insulation, acoustic insulation, filtration, use as a bacterial barrier and sterility.

These properties can be combined to produce fabrics suited for specific applications while achieving a good balance between product use-life and cost. They can mimic the appearance, texture and strength of a woven fabric and can be as bulky as the thickest paddings. In combination with other materials, they provide a spectrum of products with diverse properties.

Undergarment products of the disclosure may comprise a bamboo fabric and a non-woven material. The undergarment may include bamboo fabric and a non-woven material in addition to other materials. In some cases, the garments may principally or primarily include bamboo fabric and a non-woven material with only the addition of further components and materials to manufacture the garment including stitching, elastic components, etc.

An undergarment of the disclosure may include a central section comprising bamboo fabric and side sections comprising a non-woven material. In some cases, the garments may principally or primarily include bamboo fabric and a non-woven material with only the addition of further components and materials to manufacture the garment including stitching, elastic components, etc.

The female crotch area of the undergarment may be made with or without a sanitary napkin made out of organic and absorbent fabrics that are free of pesticides or herbicides. The pads can be ultra-thin and may be made out of plant based natural fibers that are absorbent enough to protect against menstrual leakages. The thickness dimensions of the liner/sanitary pad may range from with 150 mm to 300 mm in thickness. Some examples of fabrics that the pads can be made out of include organic cotton and bamboo. The padding/sanitary pad may or may not contain a top sheet. A top sheet may be a polyethylene/polypropylene non-woven fabric bearing an emollient finish where the core is comprised of a multi-layer, preferably two-layer, low density open-celled, polyacrylate polymer foam. A back sheet of the top layer may include or consist of an impermeable, optionally pigmented, polyethylene film.

Undergarment styles of the disclosure include men's bikini style, boxer, briefs, thong, trunks, and women's, high waisted, bikini, thong, boy briefs, hipster, bikini boxers, and g string among others.

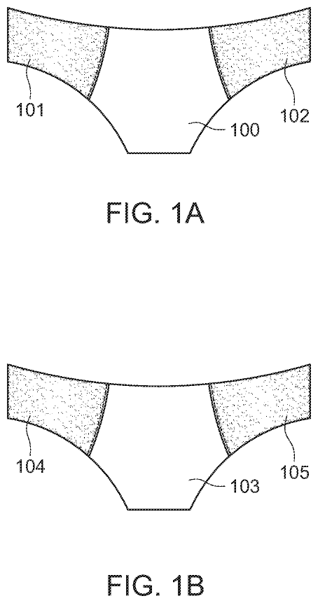

FIG. 1A shows the front of a generic undergarment of the disclosure. In. FIG. 1A, the undergarment can be understood to be roughly broken into three main areas 101, 100, and 102. The relative sizes of each of these areas or sections can vary from garment to garment. Area 100 is a central section which is connected to two side sections 101 and 102. The central section 100 may comprise bamboo fabric either entirely or in part. The side sections may comprise a nonwoven fabric either entirely or in part. For example, either side section may comprise panels that cover a part of the areas 101 and 102 or the entire respective area. A variety of side panel geometric shapes and patterns may be used in areas 101 or 102 or both.

FIG. 1B shows the back of the undergarment shown in FIG. 1A. As with the front, the back of the garment comprises three areas: 103, 104, and 105. The relative areas of these sections can again vary from garment to garment. The central section 103 may comprise bamboo fabric either entirely or in part. The side sections may again comprise a nonwoven fabric either entirely or in part. As with the front, either side section may comprise panels that cover a part of the areas 104 and 105 or the entire respective area. The side sections 105 and 105 may connect with the side sections 101 and 102 shown in FIG. 1A. For example, the respective front and back side sections may integrally connect. A variety of geometric shapes and sizes and patterns can be used for the side panels in areas 103 or 105 or both. In one preferred embodiment substantially, the entire back is bamboo fabric.

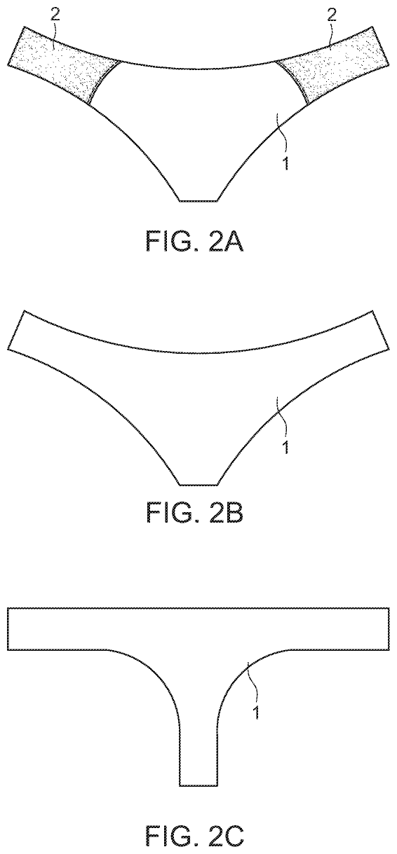

FIG. 2A shows one embodiment of an undergarment of the disclosure. In FIG. 2A, the front of the undergarment is shown including a bamboo fabric 1 and a nonwoven section 2. The nonwoven section may be a biodegradable, clothlike, material. FIG. 2B shows the back of the undergarment shown in FIG. 2A. In FIG. 2B the back includes bamboo fabric 1. FIG. 2C shows the back of a bikini style undergarment including Bamboo fabric 1.

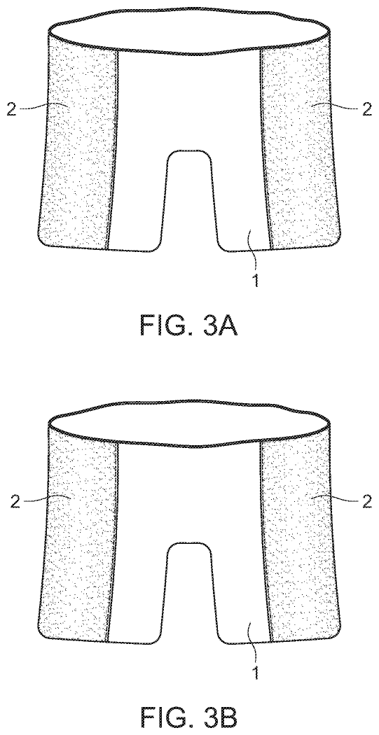

FIG. 3A shows the front of a boxer style undergarment with a bamboo fabric central section 1 and a non-woven section 2. FIG. 3B shows the back of a boxer style garment with a bamboo fabric central section 1 and a side section 2 made of nonwoven material.

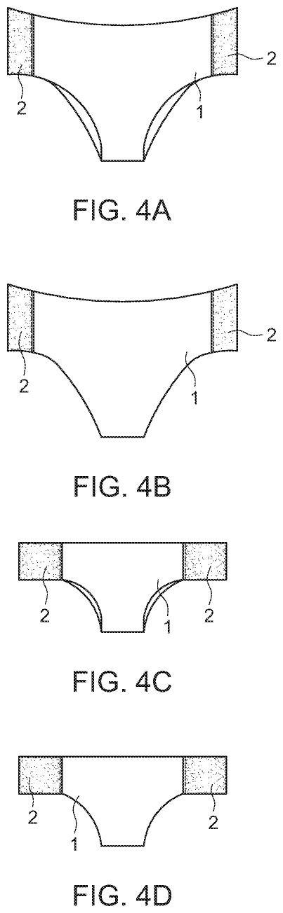

FIG. 4A shows the front of a high waist style undergarment with a bamboo fabric central section 1 and a side section 2 made of nonwoven material. FIG. 4B shows the back of a high waisted style undergarment with a bamboo fabric central section 1 and a side section 2 made of nonwoven material. FIG. 4C shows the front of a women's boy briefs style undergarment with a bamboo fabric central section 1 and a side section 2 made of nonwoven material. FIG. 4D shows the back of a women's boy-briefs style undergarment with a bamboo fabric central section 1 and a side section 2 made of nonwoven material.

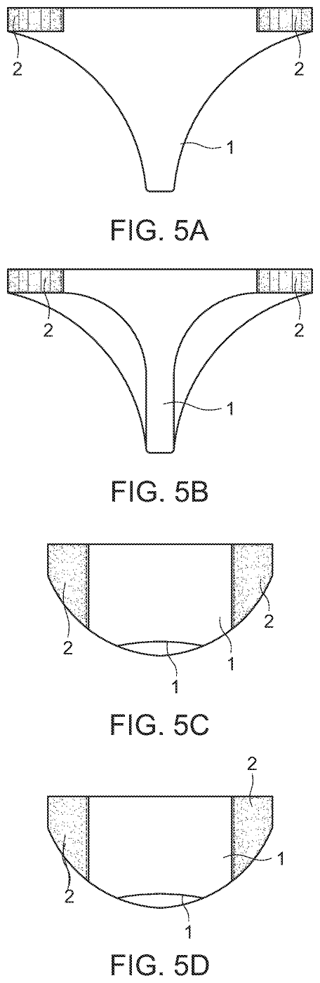

FIG. 5A shows the front of a men's thong style undergarment with a bamboo fabric central section 1 and a side section 2 made of nonwoven material. FIG. 5B shows the back of a men's thong style undergarment with a bamboo fabric central section 1 and a side section 2 made of nonwoven material. FIG. 5C shows the front of a women's bikini style undergarment with a bamboo fabric central section 1 and a side section 2 made of nonwoven material. FIG. 5D shows the back of a women's bikini style undergarment with a bamboo fabric central section 1 and a side section 2 made of nonwoven material.

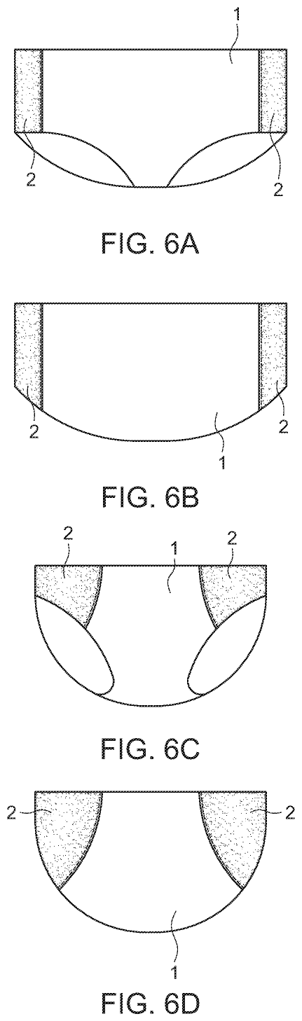

FIG. 6A shows the front of a women's hipster style undergarment with a bamboo fabric central section 1 and a side section 2 made of nonwoven material. FIG. 6B shows the back of a women's hipster style undergarment with a bamboo fabric central section 1 and a side section 2 made of nonwoven material. FIG. 6C shows the front of a men's bikini style undergarment with a bamboo fabric central section 1 and a side section 2 made of nonwoven material. FIG. 6D shows the back of a men's bikini style undergarment with a bamboo fabric central section 1 and a side section 2 made of nonwoven material.

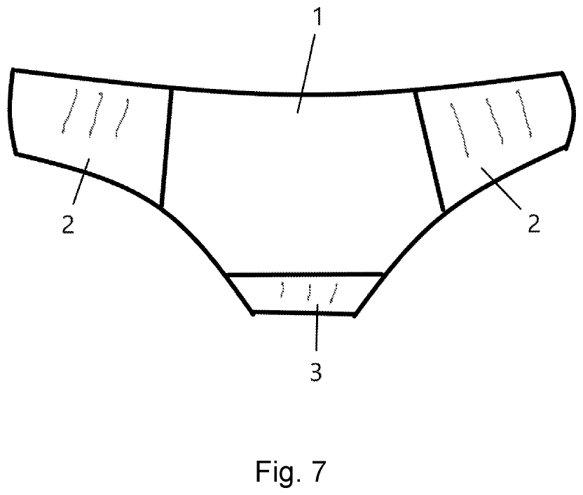

FIG. 7 shows an embodiment of an undergarment with a protective layer that may be used to protect against leakages. In FIG. 7, section 1 comprises bamboo fabric, the two side sections 2 comprise a non-woven material and bottom section 3 comprises a protective layer.

While this disclosure includes specific examples, it will be apparent after an understanding of the disclosure of this application has been attained that various changes in form and details may be made in these examples without departing from the spirit and scope of the claims and their equivalents.

What is claimed is:

1. An undergarment comprising:
a fabric comprising bamboo; and
a nonwoven material.

2. The undergarment of claim 1, comprising:
a central section comprising bamboo fabric;
two side sections, one on either side of the central section, each side section comprising a nonwoven material;
wherein the central section and the side sections are attached.

3. The undergarment of claim 2, wherein the entire back of the undergarment comprises bamboo fabric.

4. The undergarment of claim 2, wherein the nonwoven material comprises cotton, rayon, bamboo, hemp, and/or silk.

5. The undergarment of claim 2, wherein the bamboo fabric comprises about 100 grams per square meter bamboo fabric to about 150 grams per square meter bamboo fabric.

6. The undergarment of claim 2, wherein the bamboo fabric comprises about 115 grams per square meter bamboo fabric to about 135 grams per square meter bamboo fabric.

7. The undergarment of claim 2, wherein the bamboo fabric comprises about 125 grams per square meter bamboo fabric

8. The undergarment of claim 2, wherein the nonwoven fabric comprises cotton.

9. The undergarment of claim 2, wherein the bamboo fabric comprises organic bamboo.

10. The undergarment of claim 2, wherein the central section and a side section are attached by stitching.

11. The undergarment of claim 2, wherein the undergarment is seamless.

12. The undergarment of claim 2, wherein the nonwoven material is dry laid, air laid, spun bound, spun laced, and/or wet laid.

13. The undergarment of claim 1, wherein the bamboo fabric further comprises cotton, hemp, silk, wool, Tencel, lyocell/modal jute, ramie, and/or abaca.

14. The undergarment of claim 2, further comprising a section comprising a protective layer.

15. The undergarment of claim 14, wherein the protective layer comprises a sanitary napkin.

16. The undergarment of claim 15, wherein the sanitary napkin comprises a top sheet.

17. The undergarment of claim 16, wherein the top sheet comprises polyethylene and/or polypropylene non-woven fabric wherein a core is comprised of a multi-layer polymer foam.

18. The undergarment of claim 16, further comprising a back sheet comprising an impermeable polyethylene film.

19. The undergarment of claim 16, wherein the thickness of the sanitary pad ranges from about 150 mm to about 300 mm.

20. The undergarment of claim 17, wherein the polymer foam is a polyacrylate foam.
